(19) United States
(12) Patent Application Publication
(10) Pub. No.: US 2025/0042950 A1
(43) Pub. Date: Feb. 6, 2025

(54) RECOMBINANT PROTEIN FOR DISPLAYING S PROTEIN OF SARS-COV-2, RECOMBINANT VIRION, AND USE THEREOF

(71) Applicant: ZHEJIANG DIFFERENCE BIOLOGICAL TECHNOLOGY CO., LTD, Hangzhou (CN)

(72) Inventors: Ruiting CHEN, Hangzhou (CN); Huimin SUN, Hangzhou (CN); Shuihua MAO, Hangzhou (CN); Mengyun ZHOU, Hangzhou (CN); Jiasheng SONG, Hangzhou (CN)

(73) Assignee: ZHEJIANG DIFFERENCE BIOLOGICAL TECHNOLOGY CO., LTD, Hangzhou (CN)

(21) Appl. No.: 18/719,846

(22) PCT Filed: Feb. 16, 2023
(86) PCT No.: PCT/CN2023/076443

§ 371 Date: Jun. 14, 2024

(30) Foreign Application Priority Data
Dec. 16, 2021 (CN) 202111541851.3

(51) Int. Cl. C07K 14/005; A61K 39/00; A61K 39/215; A61P 31/14; C12N 7/00; C12N 15/86

(52) U.S. Cl. CPC C07K 14/005; A61K 39/215; A61P 31/14; C12N 7/00; C12N 15/86; A61K 2039/5256; C07K 2319/03; C12N 2760/18121; C12N 2760/18122; C12N 2760/18143; C12N 2770/20022; C12N 2770/20034

(57) ABSTRACT

A recombinant protein for displaying an S protein of severe acute respiratory syndrome coronavirus 2 (SARS-COV-2), a recombinant virion, and a use thereof, as well as a recombinant gene expressing the S protein of the SARS-COV-2 are provided. The recombinant gene or the recombinant protein includes a sequence of an extracellular domain of the S protein of the SARS-COV-2 and sequences of a TMD and a CTD of an F protein of an avian paramyxovirus (APMV). The recombinant Newcastle disease virus (NDV) including this sequence has a strong ability to produce a neutralizing antibody. A recombinant gene or protein produced through the recombinant of a sequence encoding the extracellular domain of the S protein and sequences encoding the TMD and the CTD of the F protein of the APMV (except for NDV) has a great potential to become an excellent antigen candidate for Coronavirus Disease 2019 (COVID-19) vaccines.

### CROSS REFERENCE TO THE RELATED APPLICATIONS

This application is the national phase entry of International Application No. PCT/CN2023/076443, filed on Feb. 16, 2023, which is based upon and claims priority to Chinese Patent Application No. 202111541851.3, filed on Dec. 16, 2021, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBHZZJ015_Sequence_Listing.xml, created on 03/15/2024, and is 71,991 bytes in size.

### TECHNICAL FIELD

The present disclosure belongs to the technical field of biological products, and specifically relates to a recombinant protein for displaying an S protein of severe acute respiratory syndrome coronavirus 2 (SARS-COV-2), a recombinant virion, and a use thereof.

### BACKGROUND

The spike of SARS-COV-2 which belongs to beta coronavirus is weakly immunogenic, when developing its vaccine using a live virus vector, ideally, it is hoped that more antigenic proteins will be displayed on the surface of the recombinant virion or expressed on the cellular membrane of the cells infected by the recombinant virus. Spike (S) protein is a key protein which binds with the hACE2 receptor on the surface of host cell and helps the virus infecting the host cell. The S protein belongs to type I transmembrane glycoprotein, and the expression level of the S protein on a cell membrane is related to the arrangement of amino acids in the transmembrane domain (TMD). Previous studies have shown that a recombinant NDV-COVS-F virus, obtained by replacing the TMD and the cytoplasmic domain (CTD) of the S protein with a TMD and a CTD of the Fusion (F) protein of Newcastle disease virus (NDV) and then rescuing, has slightly-improved immunogenicity compared with a recombinant virus obtained by directly inserting an original S protein into an NDV vector. However, replacing the TMD and the CTD of the S protein with the TMD and the CTD of the F protein of the NDV is likely to cause a competitive relationship between the insertion into a cell membrane of the S protein and the insertion into a cell membrane of the F protein of the NDV and interfere with the expression of an antigenic protein or the virus assembly on a cell membrane, thereby affecting the immunogenicity.

### SUMMARY

In view of this, an objective of the present disclosure is to provide a recombinant protein for displaying an S protein of SARS-COV-2, a recombinant virus constructed correspondingly, and a use thereof.

The present disclosure provides a recombinant protein for displaying an S protein of SARS-COV-2, including: a sequence of an extracellular domain of the S protein of the SARS-COV-2 and sequences of a TMD and a CTD of an F protein of an avian paramyxovirus (APMV).

Preferably, the APMV includes one or more of the following serotypes: APMV-2, APMV-3, APMV-4, APMV-5, APMV-6, APMV-7, APMV-8, APMV-9, APMV-10, APMV-11, and APMV-12.

Preferably, the APMV includes one or more of the following serotypes: APMV-3, APMV-5, APMV-7, and APMV-8.

Preferably, the extracellular domain of the S protein of the SARS-COV-2 is derived from an original novel coronavirus strain or a variant thereof.

Preferably, the sequence of the extracellular domain of the S protein of the SARS-COV-2 further includes one or more of the following protein-coding sequences: S2P, S6P, and S2P/GSAS:
the S2P is a protein-coding sequence produced through K986P and V987P mutations to the extracellular domain of the S protein of the SARS-COV-2;
the S2P/GSAS is a protein-coding sequence produced by mutating amino acids RRAR at positions 682 to 685 of the S2P into GSAS;
the S6P is a protein-coding sequence produced through F817P, A892P, A899P, and A942P mutations to the S2P.

The present disclosure provides recombinant genes encoding the recombinant protein.

The present disclosure provides a recombinant virus vector including the recombinant gene.

Preferably, the recombinant virus vector includes an NDV vector as a backbone vector.

Preferably, the recombinant gene is inserted between a P gene and an M gene of the NDV vector in a form of an expression cassette.

The present disclosure provides a recombinant virion or vaccine strain prepared from the recombinant virus vector.

The present disclosure provides a use of the recombinant protein, the recombinant gene, the recombinant virus vector, or the recombinant virion or vaccine strain in preparation of a vaccine for prevention and control of COVID-19.

The recombinant protein for displaying the S protein of the SARS-COV-2 provided in the present disclosure includes a sequence of an extracellular domain of the S protein of the SARS-COV-2 and a sequence encoding the TMD and the CTD from APMV F protein. In the present disclosure, the original TMD and CTD of an S protein of SARS-COV-2 are replaced with a TMD and a CTD of an F protein from the APMV to produce a recombinant protein, which can solve the following technical problems: 1) It may help avoiding a competitiveness when virus enveloping between the recombinant protein and the F protein of NDV. 2) It can improve neutralizing antibodies production, reduce non-neutralizing antibodies ratio, and greatly enhance the immunogenicity.

Further, the present disclosure specifically limits a type of APMV, where recombinant proteins S2P-AP3 (aa3), S2P-AP5, S2P-AP7, and S2P-AP8 or coding genes therefor all have excellent immunogenicity and can be used for preparation of a vaccine for prevention of COVID-19.

### BRIEF DESCRIPTION OF THE DRAWINGS

FIG. 1 is a schematic diagram of a protein expressed by the recombinant gene provided in the present disclosure, where ECD represents extracellular domain, TMD represents transmembrane domain, and CTD represents cytoplasmic domain;

FIG. 2 shows relative S1 IgG contents in immune serum detected by enzyme-linked immunosorbent assay (ELISA) in an embodiment of the present disclosure; and

FIG. 3 shows results of the SARS-CoV-2 pseudovirus neutralization assay in an embodiment of the present invention, where an x-coordinate is a serum dilution ratio and a y-coordinate is a fluorescence percentage after cells are infected with the pseudovirus.

### DETAILED DESCRIPTION OF THE EMBODIMENTS

The present disclosure provides a recombinant protein for displaying an S protein of SARS-COV-2, including: a sequence of an extracellular domain of the S protein of the SARS-COV-2 and sequences of a TMD and a CTD of an F protein of an APMV. A schematic structural diagram of the recombinant protein is shown in FIG. 1. The sequence of the extracellular domain of the S protein of the SARS-COV-2 is fused with the sequences of the TMD and the CTD of the F protein from the APMV.

In the present disclosure, the APMV preferably includes one or more of the following serotypes: APMV-2, APMV-3, APMV-4, APMV-5, APMV-6, APMV-7, APMV-8, APMV-9, APMV-10, APMV-1l, and APMV-12, and more preferably includes one or more of the following serotypes: APMV-3, APMV-5, APMV-7, and APMV-8. Source and sequence information of F proteins of APMVs is shown in Table 1.

TABLE I
Source and sequence information of F proteins of APMVs

| | | Abbreviation of a | |
|---|---|---|---|
| | | recombinant sequence | |
| | | including a TMD and a | |
| APMV | Source strain | CTD of an F protein | Sequence |
| APMV-2 | APMV-2/Procarduelis | AP2(1) | SEQ ID NO: 1 |
| | nipalensis/China/Suiling/53/2013 | | |
| | APMV-2/Chicken/England/7702/06 | AP2(2) | SEQ ID NO: 2 |
| APMV-3 | APMV3/PKT/Netherland/449/75 | AP3(aa3) | SEQ ID NO: 3 |
| | | AP3(aa33) | SEQ ID NO: 4 |
| APMV-4 | APMV- 4/White-Fronted | AP4 | SEQ ID NO: 5 |
| | Goose/Syvaske/Ukraine/6-15- | | |
| | 03/2014 | | |
| APMV-5 | Avian paramyxovirus 5 strain | AP5 | SEQ ID NO: 6 |
| | Budgerigar/Kunitachi/74 | | |
| APMV-6 | Avian paramyxovirus 6 isolate | AP6 | SEQ ID NO: 7 |
| | teal/Novosibirsk | | |
| | region/455/2009 | | |
| APMV-7 | APMV-7/dove/Tennessee/4/75 | AP7 | SEQ ID NO: 8 |
| APMV-8 | Avian paramyxovirus 8 strain | AP8 | SEQ ID NO: 9 |
| | goose/Delaware/1053/76 | | |

AP3 (aa3) is different from AP3 (aa33) in that different numbers of amino acids are truncated before a TMD of an F protein, where for the AP3 (aa3), 3 amino acids (an extracellular domain of the F protein) are included before the TMD of the F protein; and for the AP3 (aa33), 33 amino acids (the extracellular domain of the F protein) are included before the TMD of the F protein.

In the present disclosure, the S protein of the SARS-COV-2 preferably includes an S protein of SARS-COV-2 of an original strain or a variant thereof, or a mutated S protein produced through an S2P, S6P, or S2P/GSAS mutation to the S protein. In an embodiment of the present disclosure, the S protein of the SARS-COV-2 is derived from a Delta variant of SARS-COV-2.

Source and sequence information of S proteins of SARS-COV-2 is shown in Table 2.

TABLE 2
Source and sequence information of S proteins

| S protein type | Mutation type | Sequence |
|---|---|---|
| Delta S protein | — | SEQ ID NO: 13 |
| S2P | K986P and V987P mutations to the | SEQ ID NO: 10 |
| | S protein | |
| S6P | F817P, A892P, A899P, and A942P | SEQ ID NO: 11 |
| | mutations to the S2P protein | |
| S2P-GSAS | Mutating amino acids 682 to 685 | SEQ ID NO: 12 |
| | of the S2P protein from RRAR to | |
| | GSAS | |

In the present disclosure, the sequence of the extracellular domain of the S protein of the SARS-COV-2 is linked to the sequences of the TMD and the CTD of the F protein of the APMV preferably through a linker. The present disclosure has no special restriction on a sequence of the protein linker, and a protein linker well known in the art can be adopted. An amino acid sequence of the linker is preferably shown in SEQ ID NO: 14 (GGSGS).

The present disclosure provides recombinant genes encoding the recombinant protein. A sequence of the recombinant gene is shown in Table 3.

| Abbreviation of the | Corresponding amino acid | |
|---|---|---|
| recombinant gene | sequence composition | Sequence No. |
| S2P-AP2(1) | S2P sequence + AP2(1) | SEQ ID NO: 15 |
| | sequence | |
| S2P-AP2(2) | S2P sequence + AP2(2) | SEQ ID NO: 16 |
| | sequence | |
| S2P-AP3 (aa3) | S2P sequence + AP3 (aa3) | SEQ ID NO: 17 |
| | sequence | |
| S2P-AP3 (aa33) | S2P sequence + AP3 (aa33) | SEQ ID NO: 18 |
| | sequence | |
| S2P-AP4 | S2P sequence + AP4 sequence | SEQ ID NO: 19 |
| S2P-AP5 | S2P sequence + AP5 sequence | SEQ ID NO: 20 |
| S2P-AP6 | S2P sequence + AP6 sequence | SEQ ID NO: 21 |
| S2P-AP7 | S2P sequence + AP7 sequence | SEQ ID NO: 22 |
| S2P-AP8 | S2P sequence + AP8 sequence | SEQ ID NO: 23 |

In the present disclosure, the recombinant gene is prepared as follows: codon optimization is conducted, then a kozak sequence (GCCACC) is added to a 5′ terminus, and gene synthesis is conducted.

The present disclosure provides a recombinant virus vector including the recombinant gene. A backbone vector for the recombinant virus vector is preferably an NDV Lasota vector. The recombinant gene is inserted between a P gene and an M gene of the NDV vector in a form of an expression cassette.

In the present disclosure, a construction method of the recombinant NDV vector includes: a transcription termination signal (SEQ ID NO: 24, TTAGAAAAAA) and a transcription initiation signal (SEQ ID NO: 25, ACGGGTAGAA) are added to the recombinant gene, and then the recombinant gene is preferably cloned into the backbone vector through homologous recombination to obtain the recombinant virus vector.

The present disclosure provides a recombinant virion or vaccine strain prepared from the recombinant virus vector.

In the present disclosure, a plasmid expressing the recombinant protein (a backbone vector is a pcDNA3.1(−) vector) is intramuscularly injected or the recombinant virion or vaccine strain is nasally instilled to immunize an animal, and a content of the binding antibodies in serum and a neutralization ability for a SARS-CoV-2 pseudovirus are detected. Results show that: 1) TMDs and CTDs of F proteins derived from viruses of different serotypes may indirectly affect the immunogenicity of the recombinant protein. Compared with the S2P protein, the replacement of sequences of a TMD and a CTD of an F protein of an APMV can improve the neutralizing antibody level of a recombinant NDV.

2) Immunizations with recombinant proteins which the TMD and CTD from two viral strains of a same serotype may lead to inconsistent antibody levels. For example, immunizations with plasmids respectively carrying sequences of TMDs and CTDs of F proteins derived from two different viral strains of APMV-2 lead to different binding antibody production capacities.

3) Immunizations with plasmids of pS2P-AP3 (aa3), pS2P-AP7, pS2P-AP5, and pS2P-AP8 allow lower binding antibody production capacities than pS2P, but allow higher serum neutralizing antibody levels than pS2P. When a recombinant virus is used for immunization, NDV-S2P-AP7 allows a comparable binding antibody level to NDV-S2P, but exhibits a better serum neutralizing antibody level than NDV-S2P. It can be known that there is a high proportion of virus neutralizing antibodies among binding antibodies produced in the recombinant protein or recombinant NDV-immunized mice produced in the present disclosure, which lays a foundation for the development of a vaccine and the reduction of occurrence of an antibody-dependent enhancement effect during multiple immunizations in the future.

Based on the excellent immunogenicity of the recombinant protein, the present disclosure provides a use of the recombinant virus vector or the recombinant virion or vaccine strain in preparation of a vaccine for prevention and control of COVID-19.

The present disclosure has no special restriction on a preparation method of the vaccine, and a vaccine preparation method well known in the art can be adopted. The vaccine has a strong ability to produce the neutralizing antibody, and thus has a high application value in prevention and control of COVID-19. The vaccine preferably includes the recombinant virion or vaccine strain and an adjuvant.

The recombinant protein and recombinant virus for displaying an S protein of SARS-COV-2 and the use thereof provided by the present disclosure are described in detail below in conjunction with examples, but these examples may not be understood as a limitation to the protection scope of the present disclosure.

### Example 1

### 1. Design and Synthesis of Recombinant Genes

A coding sequence of an extracellular domain (amino acids 1 to 1213, SEQ ID NO: 17) of an S protein of SARS-COV-2 (a Delta variant of SARS-COV-2) was subjected to K986P and V987P mutations, and then fused with a coding sequence of a part of a C terminus including the TMD and CTD of F proteins from serotypes APMV-2, APMV-3, APMV-4, APMV-5, APMV-6, APMV-7, and APMV-8 respectively. A protein linker was added between the coding sequence of above S2P protein and a coding sequence of a part of a C terminus of an F protein including TMD and CTD. Because F proteins of APMVs except for NDV were not included in the Swiss-Prot database, a TMD of an F protein of a virus strain selected from APMV-2, APMV-3, APMV-4, APMV-5, APMV-6, APMV-7, and APMV-8 was predicted with the TMD prediction software TMHMM-2.0. It should be noted that prediction results of the TMD and the CTD obtained by different protein structure prediction software may be different in several amino acids, which is within the protection scope of the present disclosure. An APMV-2 F gene was derived from a genome of a virus strain APMV-2/Procarduelis nipalensis/China/Suiling/53/2013 or APMV-2/Chicken/England/7702/06, and S2P-AP2 (1) and S2P-AP2 (2) recombinant gene sequences were designed, respectively. An APMV-3 F gene was derived from a genome of a virus strain APMV3/PKT/Netherland/449/75, and S2P-AP3 (aa3) and S2P-AP3 (aa33) recombinant gene sequences were designed, respectively. An APMV-4 F gene was derived from a genome of a virus strain APMV-4/White-Fronted Goose/Syvaske/Ukraine/6-15-03/2014, and an S2P-AP4 recombinant gene sequence was designed. An APMV-5 F gene was derived from a genome of a virus strain Avian paramyxovirus 5 strain Budgerigar/Kunitachi/74, and an S2P-AP5 recombinant gene sequence was designed. An APMV-6 F gene was derived from a genome of a virus strain Avian paramyxovirus 6 isolate teal/Novosibirsk region/455/2009, and an S2P-AP6 recombinant gene sequence was designed. An APMV-7 F gene was derived from a genome of a virus strain APMV-7/dove/Tennessee/4/75, and an S2P-AP7 recombinant gene sequence was designed. An APMV-8 F gene was derived from a genome of a virus strain Avian paramyxovirus 8 strain goose/Delaware/1053/76, and an S2P-AP8 recombinant gene sequence was designed.

After the recombinant gene sequences were designed, codon optimization was conducted, then a kozak sequence (GCCACC) was added at the 5′ terminus, and gene synthesis was entrusted by GENEWIZ. Each synthesized recombinant gene sequence was cloned between EcoRI and XhoI restriction enzyme cleavage sites of a pcDNA3.1(+) vector to produce expression plasmids pS2P-AP2 (1), pS2P-AP2 (2), pS2P-AP3 (aa3), pS2P-AP3 (aa33), pS2P-AP4, pS2P-AP5, pS2P-AP6, pS2P-AP7, and pS2P-AP8.

### Example 2

### Construction of Recombinant NDV Vectors and Rescue of Recombinant NDVs

A transcription termination signal sequence (TTAGAAAAAA, SEQ ID NO: 24) was added at the 3′ terminus of each recombinant gene sequence designed in Example 1, and a transcription initiation signal sequence (ACGGGTAGAA, SEQ ID NO: 25) was added at the 5′ terminus of each recombinant gene sequence. Primers were designed to amplify each recombinant gene sequence including the transcription initiation signal and the transcription termination signal, and each recombinant gene sequence was inserted between the P and M gene of NDV Lasota vector through homologous recombination to obtain a series of recombinant NDV vectors.

Each of the recombinant NDV vectors was mixed with virus rescue helper plasmids pCI-NP, pCI-P, and pCI-L in a ratio of 1:1:1:1, the plasmid concentration was determined by Nano drop, and each plasmid mixture was transfected into BHK21-T7 cells. 72 h after the transfection, the cells were repeatedly frozen and thawed 3 times, a resulting lysate was inoculated into 9 to 11-day-old SPF chicken embryos at 0.2 mL/embryo to 0.3 mL/embryo, and the chicken embryos were cultivated at 37° C. for 24 h. Dead embryos within 24 h were discarded, and an allantoic fluid was collected from each survival chicken embryo within 72 h and tested for a hemagglutination titer. An allantoic fluid with a hemagglutination titer of 2 log or more of a survival chicken embryo was centrifuged, dispensed, and stored in a −80° C. refrigerator, and extracted RNA was reverse-transcribed according to instructions of a HiScript III 1st Strand cDNA Synthesis Kit to synthesize first-strand cDNA. Then synthesized cDNA was identified by PCR gel electrophoresis. The PCR system and conditions were set according to requirements in instructions of a HiscriptII One Step RT-PCR kit of Vazyme. For the identification, an upstream primer NDV-F3153: AAGGTCCAACTCTCCAAGCGG (SEQ ID NO: 26), and a downstream primer NDV-R3454: GTCCTCCTTACTATCAGTCCACA (SEQ ID NO: 27). Recombinant NDV vaccine strains finally produced were named NDV-S2P-AP3 (aa3) and NDV-S2P-AP7, respectively.

Sequences of ECD of S2P protein were fused with sequences of the TMD and CTD of F proteins of NDV to obtain a recombinant protein (SEQ ID NO: 28). A recombinant gene (SEQ ID NO: 29) encoding the recombinant protein was synthesized according to the method in Example 1. A recombinant vector and a rescued virus were constructed according to the methods in Example 2. A recombinant NDV virion finally obtained was named NDV-S2P/F34.

### Example 3

### Mouse Immunization Test

4 to 5-week-old Babl/c mice were immunized with the recombinant gene-containing plasmid constructed in Example 1 and the recombinant NDV constructed in Example 2, where four mice were immunized with the plasmid or the recombinant virus. An immunization program for the plasmid was as follows: The plasmid was intramuscularly injected at 100 μg/mouse (sterile PBS could be supplemented, and a final injection volume was no less than 100 μL/mouse). 10 days after immunization, blood was collected. An immunization program for the recombinant NDV was as follows: The recombinant NDV was nasally instilled at 50 μL/mouse. The immunization was conducted on day 0 and day 7, and blood was collected on day 14. A hemagglutination titer of the recombinant virus was 6 log 2 to 7 log 2 during immunization. In the control group, mice were immunized with sterile PBS.

### Example 4

### Detection of an Antibody Binding to an S Protein of SARS-COV-2 in Immune Serum by ELISA

A 96-well ELISA plate was equilibrated at room temperature in advance, and a serum sample and a positive control each were diluted 20-fold with a diluent in a SARS-COV-2(2019-nCoV) Spike S1 antibody titer test kit of Sino Biological. Plate washing, sample incubation, antibody incubation, a chromogenic reaction, and chromogenic reaction termination were conducted according to requirements in instructions of the test kit, and finally an OD₄₅₀ value was read with a microplate reader. An OD₄₅₀ reading for each sample was divided by a background OD₄₅₀ reading to obtain relative IgG antibody data.

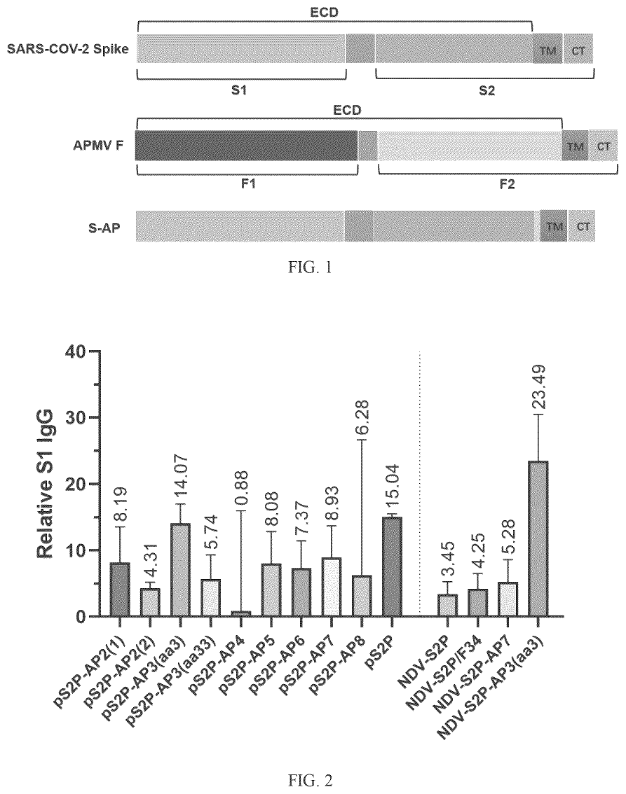

Results were shown in FIG. 2. It can be seen that, after mice were immunized, plasmids carrying recombinant genes S2P-AP3 (aa3), S2P-AP5, S2P-AP7, and S2P-AP8 and recombinant NDVs NDV-S2P-AP3 (aa3) and NDV-S2P-AP7 allow relatively-high binding antibody levels. In particular, NDV-S2P-AP3 (aa3) allows an antibody level that is about 7 times higher than antibody level of NDV-S2P. However, if a plasmid allows a high binding antibody level, recombinant NDV produced from the plasmid does not necessarily allow a high binding antibody level, which may be related to the budding and packaging mechanism of a virus. It can be seen from experimental results that two virus strains of the same serotype APMV-2 show inconsistent antibody levels, indicating that the immunization result of a recombinant protein produced by fusing an extracellular domain of S protein with the TMD and CTD of F protein of a virus strain may not fully reflect immune effects of all other virus strains of the same serotype. Given that there are too many virus strains of the APMV family, there is still a broad space for in-depth research here. In addition, immunization effects of pS2P-AP3 (aa3) and pS2P-AP3 (aa33) are quite different, and amino acid sequences of TMDs and CTDs of proteins expressed by the two recombinant genes are the same, except that extension fragments selected before the TMDs have different lengths. It is speculated that this result is very likely due to the fact that the selected extension fragment before a TMD affects a conformation of the entire recombinant protein.

### Example 5

### Pseudovirus Neutralization Assay

15 μL of serum to be tested was added to a 96-well plate, and then 135 μL of DMEM was added for dilution. 2-fold dilution was conducted with 4 gradients. 50 μL of a pseudovirus solution was added to diluted serum and incubated at 37° C. for 1 h. A pseudovirus was VSV-SΔ 24-GFP (Xiong H L, Wu Y T, Cao J L, et al. Robust neutralization assay based on SARS-COV-2 S-protein-bearing vesicular stomatitis virus (VSV) pseudovirus and ACE2-overexpressing BHK21 cells. Emerg Microbes Infect. 2020; 9 (1): 2105-2113.doi: 10.1080/22221751.2020.1815589), and was constructed as follows: the G protein was deleted from the VSV vector, then an S A 24 gene of SARS-CoV-2 and a GFP gene were inserted into the VSV vector, and then virus rescue was conducted, where the SΔ24 gene deleted 24 amino acids at a C terminus of S protein, such that the S protein could be increasingly presented on the envelope of VSV. When the incubation was conducted for 30 min, Vero-E6 cells were digested. After the incubation was completed, 100 μL of the cells was added to each well and cultivated in an incubator at 37° C. and 5% CO₂ for 24 h. A serum-free cell control (150 μL of DMEM+100 μL of the cells) and a serum-free virus control (100 μL of DMEM+50 μL of the pseudovirus+100 μL of the cells) were set. Finally, the plate was photographed under a fluorescence microscope, and a fluorescence percentage of cells in each well was recorded.

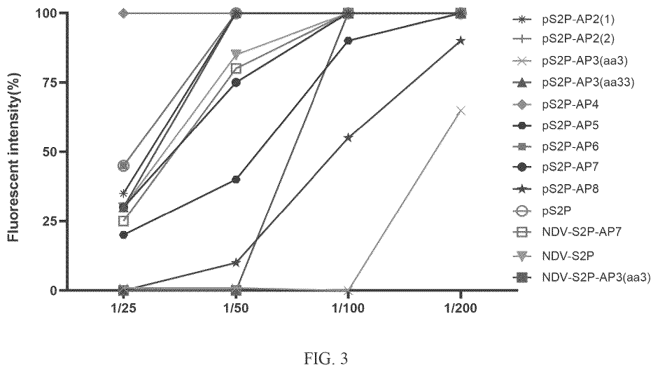

Results were shown in FIG. 3 and Table 4.

TABLE 4
Neutralization assay results of plasmids and
recombinant NDVs carrying recombinant genes

Serum dilution ratio

| Plasmid/virus | 1/25 | 1/50 | 1/100 | 1/200 |
|---|---|---|---|---|
| pS2P-AP2(1) | 35% | 100% | 100% | 100% |
| pS2P-AP2(2) | 100% | 100% | 100% | 100% |
| pS2P-AP3(aa3) | 1% | 1% | 0% | 65% |
| pS2P-AP3(aa33) | 30% | 100% | 100% | 100% |
| pS2P-AP4 | 100% | 100% | 100% | 100% |
| pS2P-AP5 | 20% | 40% | 90% | 100% |
| pS2P-AP6 | 45% | 100% | 100% | 100% |
| pS2P-AP7 | 30% | 75% | 100% | 100% |
| pS2P-AP8 | 0 | 10% | 55% | 90% |
| pS2P | 45% | 100% | 100% | 100% |
| NDV-S2P-AP3(aa3) | 0 | 0 | 100% | 100% |
| NDV-S2P-AP7 | 25% | 80% | 100% | 100% |
| NDV-S2P | 30% | 85% | 100% | 100% |

It can be seen from FIG. 3 that serum neutralizing antibody levels in mice immunized with plasmids pS2P-AP3 (aa3), pS2P-AP8, pS2P-AP5, and pS2P-AP7 and NDV-S2P-AP7 are higher than a serum neutralizing antibody level in mice immunized with pS2P or NDV-S2P.

An excellent antigen should make neutralizing antibodies produced as many as possible. It can be seen from the results of the binding antibody and pseudovirus neutralizing antibody tests of the present disclosure that, during plasmid immunization, the recombinant genes S2P-AP3 (aa3), S2P-AP5, and S2P-AP7 allow a slightly-lower binding antibody level than the S2P gene, but have a stronger ability to produce neutralizing antibodies than the S2P gene. In the present disclosure, a recombinant gene with a relatively-excellent plasmid immunization effect is selected for testing during a rescue of a recombinant virus, but it cannot exclude that other recombinant genes with general plasmid immunization effects cannot allow a similar excellent immunization effect. It can be known that a recombinant gene or protein produced through the recombinant of a sequence encoding an extracellular domain of an S protein and sequences encoding the TMD and CTD of F protein of APMV (except for NDV) has a great potential to become an excellent antigen candidate for COVID-19 vaccines.

The above description of examples is merely provided to help illustrate the method of the present disclosure and a core idea thereof. It should be noted that several improvements and modifications may be made by a person of ordinary skill in the art without departing from the principle of the present disclosure, and these improvements and modifications should also fall within the protection scope of the present disclosure. Various modifications to these examples are apparent to those of professional skill in the art, and the general principles defined herein may be implemented in other examples without departing from the spirit or scope of the present disclosure. Thus, the present disclosure is not limited to the examples shown herein, but falls within the widest scope consistent with the principles and novel features disclosed herein.

What is claimed is:

1. A recombinant protein for displaying an S protein of severe acute respiratory syndrome coronavirus 2 (SARS-COV-2), comprising: a sequence of an extracellular domain of the S protein of the SARS-COV-2, a sequence of a transmembrane domain (TMD) of an F protein of an avian paramyxovirus (APMV), and a sequence of a cytoplasmic domain (CTD) of the F protein of the APMV.

2. The recombinant protein according to claim 1, wherein the APMV comprises one or more of the following serotypes: APMV-2, APMV-3, APMV-4, APMV-5, APMV-6, APMV-7, APMV-8, APMV-9, APMV-10, APMV-11, and APMV-12.

3. The recombinant protein according to claim 1, wherein the APMV comprises one or more of the following serotypes: APMV-3, APMV-5, APMV-7, and APMV-8.

4. The recombinant protein according to claim 1, wherein the extracellular domain of the S protein of the SARS-COV-2 is derived from an original SARS-COV-2 strain or a variant of the original SARS-COV-2 strain.

5. The recombinant protein according to claim 4, wherein the sequence of the extracellular domain of the S protein of the SARS-COV-2 further comprises one or more of the following protein-coding sequences: S2P, S6P, and S2P/GSAS;
the S2P is a protein-coding sequence produced through K986P and V987P mutations to the extracellular domain of the S protein of the SARS-COV-2;
the S2P/GSAS is a protein-coding sequence produced by mutating amino acids RRAR at positions 682 to 685 of the S2P into GSAS; and
the S6P is a protein-coding sequence produced through F817P, A892P, A899P, and A942P mutations to the S2P.

6. A recombinant gene encoding the recombinant protein according to claim 1.

7. A recombinant virus vector comprising the recombinant gene according to claim 6.

8. The recombinant virus vector according to claim 7, wherein the recombinant virus vector comprises a Newcastle disease virus (NDV) vector as a backbone vector.

9. The recombinant virus vector according to claim 8, wherein the recombinant gene is inserted between a P gene and an M gene of the NDV vector in a form of an expression cassette.

10. The recombinant virus vector according to claim 7, wherein the recombinant virus vector is obtained by adding a transcription initiation signal at a 5′ terminus of an S2P-AP3 (aa3) recombinant gene, an S2P-AP8 recombinant gene, an S2P-AP5 recombinant gene, or an S2P-AP7 recombinant gene, adding a transcription termination signal at a 3′ terminus of the S2P-AP3 (aa3) recombinant gene, the S2P-AP8 recombinant gene, the S2P-AP5 recombinant gene, or the S2P-AP7 recombinant gene, and cloning a resulting recombinant gene between a P gene and an M gene of an NDV Lasota vector through a homologous recombination;
the nucleotide sequence of the S2P-AP3 (aa3) recombinant gene is shown in SEQ ID NO: 17;
the nucleotide sequence of the S2P-AP8 recombinant gene is shown in SEQ ID NO: 23;
the nucleotide sequence of the S2P-AP5 recombinant gene is shown in SEQ ID NO: 20; and
the nucleotide sequence of the S2P-AP7 recombinant gene is shown in SEQ ID NO: 22.

11. A recombinant virion or vaccine strain prepared from the recombinant virus vector according to claim 7.

12. The recombinant virion or vaccine strain according to claim 11, comprising NDV-S2P-AP3 (aa3) and/or NDV-S2P-AP7;
wherein the NDV-S2P-AP3 (aa3) is obtained through a rescue of a recombinant NDV vector expressing an S2P-AP3 (aa3) recombinant gene, and the nucleotide sequence of the S2P-AP3 (aa3) recombinant gene is shown in SEQ ID NO: 17; and
the NDV-S2P-AP7 is obtained through a rescue of a recombinant NDV vector expressing an S2P-AP7 recombinant gene, and the nucleotide sequence of the S2P-AP7 recombinant gene is shown in SEQ ID NO: 22.

13. A method for preparing a vaccine for prevention and control of Coronavirus Disease 2019 (COVID-19), comprising: using the recombinant protein according to claim 1, a recombinant gene encoding the recombinant protein, a recombinant virus vector comprising the recombinant gene, or a recombinant virion or vaccine strain prepared from the recombinant virus vector.

14. A vaccine for prevention and control of COVID-19, wherein an active ingredient of the vaccine comprises the recombinant protein according to claim 1, a recombinant virus vector comprising the recombinant gene, or a recombinant virion or vaccine strain prepared from the recombinant virus vector.

15. A method for prevention and control of COVID-19, comprising: using the recombinant protein according to claim 1, a recombinant gene encoding the recombinant protein, a recombinant virus vector comprising the recombinant gene, or a recombinant virion or vaccine strain prepared from the recombinant virus vector.

16. A method for prevention and control of COVID-19, comprising: intramuscularly immunizing the recombinant virus vector according to claim 7 for an immunization or nasally immunizing a recombinant virion or vaccine strain prepared from the recombinant virus vector for the immunization.

17. The recombinant gene according to claim 6, wherein in the recombinant protein, the APMV comprises one or more of the following serotypes: APMV-2, APMV-3, APMV-4, APMV-5, APMV-6, APMV-7, APMV-8, APMV-9, APMV-10, APMV-11, and APMV-12.

18. The recombinant gene according to claim 6, wherein in the recombinant protein, the APMV comprises one or more of the following serotypes: APMV-3, APMV-5, APMV-7, and APMV-8.

19. The recombinant gene according to claim 6, wherein in the recombinant protein, the extracellular domain of the S protein of the SARS-COV-2 is derived from an original SARS-COV-2 strain or a variant of the original SARS-COV-2 strain.

20. The recombinant gene according to claim 19, wherein wherein the sequence of the extracellular domain of the S protein of the SARS-COV-2 further comprises one or more of the following protein-coding sequences: S2P, S6P, and S2P/GSAS;
the S2P is a protein-coding sequence produced through K986P and V987P mutations to the extracellular domain of the S protein of the SARS-COV-2;
the S2P/GSAS is a protein-coding sequence produced by mutating amino acids RRAR at positions 682 to 685 of the S2P into GSAS; and
the S6P is a protein-coding sequence produced through F817P, A892P, A899P, and A942P mutations to the S2P.


SEQUENCE LISTING (29 sequences)

SEQ ID NO: 1 - protein, 94 aa, unidentified
LDISSEIGSINNTVNKVEELIAESNAWLQAVNPHLVNNTSIIVLCVLAAIFVVWLVALTGCLAYYIKKSSATRMVGIGSSPAGNPYVAQSATKM

SEQ ID NO: 2 - protein, 98 aa, unidentified
IDISNELENVNSSLSSALEKLDESDAALSKVNVHLTSTSAMATYIVLTVIALILGFVGLGLGCFAMIKVKSQAKTLLWLGAHADRSYILQSKPAQSST

SEQ ID NO: 3 - protein, 58 aa, unidentified
YSNWYIVLVIVLLFGNLGWSLLLTVLLCRSRKQQRRYQQDDSVGSERGVGVGTIQYMS

SEQ ID NO: 4 - protein, 88 aa, unidentified
LIQEAQDHANAALAKVEESDKYLRAVTGGNYSNWYIVLVIVLLFGNLGWSLLLTVLLCRSRKQQRRYQQDDSVGSERGVGVGTIQYMS

SEQ ID NO: 5 - protein, 105 aa, unidentified
QIVSVDPIDISSDIAKINSSIEAAREQLELSNQILSRINPRIVNDESLIAIIVTIVVLSLLVIGLIVVLSVMYKNLKKVQRAQAAMMMQQMSSSQPVTTKLGTPF

SEQ ID NO: 6 - protein, 90 aa, unidentified
DISVELNRVNTSLSKVESLIKESNSILDSVNPQILNVKTVIILAVIIGLIVVWCFILTCLIVRGFMLLVKQQKFKGLSVQNNPYVSNNSH

SEQ ID NO: 7 - protein, 94 aa, unidentified
PLDISSELGKVNQSLSNVEDLIDQSNQLLNRVNPNIVNNTAIIVTIVLLVLLVLWCLALTISILYVSKHAVRMIKTVPNPYVMQAKSPGSATQF

SEQ ID NO: 8 - protein, 84 aa, unidentified
ELGQLNNSISSATDNLQKAQESLNKSIIPAATSSWLIILLFVLVSISLVIGCISIYFIYKHSTTNRSRNLSSDIISNPYIQKAN

SEQ ID NO: 9 - protein, 96 aa, unidentified
LDISSEIGNINNTVNRVEDLIHQSEEWLAKVNPHIVNNTTLIVLCVLSALAVIWLAVLTAIIIYLRTKLKTISALAVTNTIQSNPYVNQTKRESKF

SEQ ID NO: 10 - protein, 1211 aa, unidentified
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 11 - protein, 1211 aa, unidentified
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 12 - protein, 1211 aa, unidentified
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 13 - protein, 1211 aa, unidentified
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 14 - protein, 5 aa, synthetic construct
GGSGS

SEQ ID NO: 15 - DNA, 3936 bp, unidentified
  source 1..3936: mol_type = other DNA
atgttcgtgtttctggtgctgctgcctctggtgtccagccagtgtgtgaacctgcggaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtgtactatcacaagaacaacaagagctggatggaaagcggcgtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattaccggtacaggctgttccggaagtccaatctgaagcccttcgagcgggacatctccaccgagatctatcaggccggcagcaagccttgcaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtacagaggtgccagtggccattcacgccgatcagctgacccctacttggcgggtgtactccacaggcagcaatgtgtttcagaccagagccggctgtctgatcggagccgagcacgtgaacaatagctacgagtgcgacatccccatcggcgctggcatctgtgccagctaccagacacagaccaacagcaggcggagagccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcagaacgtcgtgaaccagaacgcccaggcactgaacaccctggtcaagcagctgtcctccaacttcggcgccatctctagcgtgctgaacgatatcctgagcagactggaccctcctgaggccgaggtgcagatcgacagactgatcacaggcagactccagtctctgcagacctacgtgacccagcagctgatcagagccgccgagattagagccagcgctaatctggccgccaccaagatgtctgagtgtgtgctgggccagagcaagagagtggacttttgcggcaagggctaccacctgatgagcttccctcagtctgcaccacacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtggtcaacatccagaaagagatcgaccggctgaacgaggtggccaagaatctgaacgagagcctgatcgacctgcaagaactggggaagtacgagcagtacatcaagtggcctggcggaagcggcagcagcctggatatctcttctgagatcggctccatcaacaacaccgtgaacaaggtggaagaactgattgccgagagcaacgcctggctgcaggccgtgaatcctcacctggtcaacaataccagcatcatcgtgctgtgcgtgctggccgctatcttcgttgtgtggctggtggctctgacaggctgtctggcctactacatcaagaagtccagcgccaccagaatggtcggaatcggcagttctcctgccggcaatccttacgtggcccagagcgccacaaaaatgtga

SEQ ID NO: 16 - DNA, 3945 bp, unidentified
  source 1..3945: mol_type = other DNA
atgttcgtgtttctggtgctgctgcctctggtgtccagccagtgtgtgaacctgcggaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtgtactatcacaagaacaacaagagctggatggaaagcggcgtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattaccggtacaggctgttccggaagtccaatctgaagcccttcgagcgggacatctccaccgagatctatcaggccggcagcaagccttgcaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtacagaggtgccagtggccattcacgccgatcagctgacccctacttggcgggtgtactccacaggcagcaatgtgtttcagaccagagccggctgtctgatcggagccgagcacgtgaacaatagctacgagtgcgacatccccatcggcgctggcatctgtgccagctaccagacacagaccaacagcaggcggagagccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcagaacgtcgtgaaccagaacgcccaggcactgaacaccctggtcaagcagctgtcctccaacttcggcgccatctctagcgtgctgaacgatatcctgagcagactggaccctcctgaggccgaggtgcagatcgacagactgatcacaggcagactccagtctctgcagacctacgtgacccagcagctgatcagagccgccgagattagagccagcgctaatctggccgccaccaagatgtctgagtgtgtgctgggccagagcaagagagtggacttttgcggcaagggctaccacctgatgagcttccctcagtctgcaccacacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtggtcaacatccagaaagagatcgaccggctgaacgaggtggccaagaatctgaacgagagcctgatcgacctgcaagaactggggaagtacgagcagtacatcaagtggcctggcggctccggcagcatcgacatctctaatgagctggaaaacgtgaacagctctctgagcagcgccctcgagaagctggatgagtctgatgccgctctgagcaaagtgaacgtgcacctgacaagcaccagcgccatggccacctatatcgtgctgaccgtgatcgccctgatcctgggctttgttggactcggcctgggctgcttcgccatgatcaaagtgaagtcccaggccaagactctgctgtggctgggagcccatgccgacagatcttacatcctgcagagcaagcccgctcagagcagcacttaa

SEQ ID NO: 17 - DNA, 3825 bp, unidentified
  source 1..3825: mol_type = other DNA
atgttcgtgtttctggtgctgctgcctctggtgtccagccagtgtgtgaacctgcggaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtgtactatcacaagaacaacaagagctggatggaaagcggcgtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattaccggtacaggctgttccggaagtccaatctgaagcccttcgagcgggacatctccaccgagatctatcaggccggcagcaagccttgcaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtacagaggtgccagtggccattcacgccgatcagctgacccctacttggcgggtgtactccacaggcagcaatgtgtttcagaccagagccggctgtctgatcggagccgagcacgtgaacaatagctacgagtgcgacatccccatcggcgctggcatctgtgccagctaccagacacagaccaacagcaggcggagagccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcagaacgtcgtgaaccagaacgcccaggcactgaacaccctggtcaagcagctgtcctccaacttcggcgccatctctagcgtgctgaacgatatcctgagcagactggaccctcctgaggccgaggtgcagatcgacagactgatcacaggcagactccagtctctgcagacctacgtgacccagcagctgatcagagccgccgagattagagccagcgctaatctggccgccaccaagatgtctgagtgtgtgctgggccagagcaagagagtggacttttgcggcaagggctaccacctgatgagcttccctcagtctgcaccacacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtggtcaacatccagaaagagatcgaccggctgaacgaggtggccaagaatctgaacgagagcctgatcgacctgcaagaactggggaagtacgagcagtacatcaagtggcctggcggctccggcagctacagcaattggtatatcgtgctggtcatcgtgctgctgttcggcaatctcggatggtcactgctgctgactgtcctgctgtgccggtccagaaagcagcagaggcggtatcagcaggacgatagcgtgggaagcgagcgcggagttggagtgggaaccatccagtacatgagctga

SEQ ID NO: 18 - DNA, 3915 bp, unidentified
  source 1..3915: mol_type = other DNA
atgttcgtgtttctggtgctgctgcctctggtgtccagccagtgtgtgaacctgcggaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtgtactatcacaagaacaacaagagctggatggaaagcggcgtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattaccggtacaggctgttccggaagtccaatctgaagcccttcgagcgggacatctccaccgagatctatcaggccggcagcaagccttgcaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtacagaggtgccagtggccattcacgccgatcagctgacccctacttggcgggtgtactccacaggcagcaatgtgtttcagaccagagccggctgtctgatcggagccgagcacgtgaacaatagctacgagtgcgacatccccatcggcgctggcatctgtgccagctaccagacacagaccaacagcaggcggagagccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcagaacgtcgtgaaccagaacgcccaggcactgaacaccctggtcaagcagctgtcctccaacttcggcgccatctctagcgtgctgaacgatatcctgagcagactggaccctcctgaggccgaggtgcagatcgacagactgatcacaggcagactccagtctctgcagacctacgtgacccagcagctgatcagagccgccgagattagagccagcgctaatctggccgccaccaagatgtctgagtgtgtgctgggccagagcaagagagtggacttttgcggcaagggctaccacctgatgagcttccctcagtctgcaccacacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtggtcaacatccagaaagagatcgaccggctgaacgaggtggccaagaatctgaacgagagcctgatcgacctgcaagaactggggaagtacgagcagtacatcaagtggcctggcggctccggcagcctgattcaagaggcccaggatcatgccaatgccgctctggccaaggtggaagagagcgacaagtacctgagagccgtgaccggcggcaattactccaactggtacatcgtgctcgtgatcgtgctgctgttcggcaatctcggatggtcactgctgctgactgtcctgctgtgccggtccagaaagcagcagaggcggtatcagcaggacgatagcgtgggaagcgagcgcggagttggagtgggaaccatccagtacatgagctga

SEQ ID NO: 19 - DNA, 3966 bp, unidentified
  source 1..3966: mol_type = other DNA
atgttcgtgtttctggtgctgctgcctctggtgtccagccagtgtgtgaacctgcggaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtgtactatcacaagaacaacaagagctggatggaaagcggcgtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattaccggtacaggctgttccggaagtccaatctgaagcccttcgagcgggacatctccaccgagatctatcaggccggcagcaagccttgcaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtacagaggtgccagtggccattcacgccgatcagctgacccctacttggcgggtgtactccacaggcagcaatgtgtttcagaccagagccggctgtctgatcggagccgagcacgtgaacaatagctacgagtgcgacatccccatcggcgctggcatctgtgccagctaccagacacagaccaacagcaggcggagagccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcagaacgtcgtgaaccagaacgcccaggcactgaacaccctggtcaagcagctgtcctccaacttcggcgccatctctagcgtgctgaacgatatcctgagcagactggaccctcctgaggccgaggtgcagatcgacagactgatcacaggcagactccagtctctgcagacctacgtgacccagcagctgatcagagccgccgagattagagccagcgctaatctggccgccaccaagatgtctgagtgtgtgctgggccagagcaagagagtggacttttgcggcaagggctaccacctgatgagcttccctcagtctgcaccacacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtggtcaacatccagaaagagatcgaccggctgaacgaggtggccaagaatctgaacgagagcctgatcgacctgcaagaactggggaagtacgagcagtacatcaagtggcctggcggaagcggcagccagatcgtgtctgtggaccctatcgacatcagctccgatatcgccaagatcaactccagcatcgaggccgccagagagcagctggaactgagcaaccagatcctgagccggatcaaccccagaatcgtgaatgacgagtccctgattgccatcatcgtgacaatcgtggtgctgtccctgctggtcatcggcctgattgtggtcctgagcgtgatgtataagaacctgaagaaggtccagcgcgcccaggccgctatgatgatgcagcagatgagcagcagccagcctgtgaccaccaagctgggcacccctttttga

SEQ ID NO: 20 - DNA, 3921 bp, unidentified
  source 1..3921: mol_type = other DNA
atgttcgtgtttctggtgctgctgcctctggtgtccagccagtgtgtgaacctgcggaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtgtactatcacaagaacaacaagagctggatggaaagcggcgtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattaccggtacaggctgttccggaagtccaatctgaagcccttcgagcgggacatctccaccgagatctatcaggccggcagcaagccttgcaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtacagaggtgccagtggccattcacgccgatcagctgacccctacttggcgggtgtactccacaggcagcaatgtgtttcagaccagagccggctgtctgatcggagccgagcacgtgaacaatagctacgagtgcgacatccccatcggcgctggcatctgtgccagctaccagacacagaccaacagcaggcggagagccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcagaacgtcgtgaaccagaacgcccaggcactgaacaccctggtcaagcagctgtcctccaacttcggcgccatctctagcgtgctgaacgatatcctgagcagactggaccctcctgaggccgaggtgcagatcgacagactgatcacaggcagactccagtctctgcagacctacgtgacccagcagctgatcagagccgccgagattagagccagcgctaatctggccgccaccaagatgtctgagtgtgtgctgggccagagcaagagagtggacttttgcggcaagggctaccacctgatgagcttccctcagtctgcaccacacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtggtcaacatccagaaagagatcgaccggctgaacgaggtggccaagaatctgaacgagagcctgatcgacctgcaagaactggggaagtacgagcagtacatcaagtggcctggcggctccggcagcgatattagcgtggaactgaaccgcgtgaacaccagcctgtctaaggtggaaagcctgatcaaagagtccaactccatcctcgacagcgtgaaccctcagatcctgaacgtgaaaaccgtgatcatcctggccgtgattatcggcctgatcgttgtgtggtgcttcatcctgacctgcctgattgtgcggggcttcatgctgctcgtgaagcagcagaagttcaagggcctgagcgtgcagaacaacccctatgtgtccaacaacagccactga

SEQ ID NO: 21 - DNA, 3933 bp, unidentified
  source 1..3933: mol_type = other DNA
atgttcgtgtttctggtgctgctgcctctggtgtccagccagtgtgtgaacctgcggaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtgtactatcacaagaacaacaagagctggatggaaagcggcgtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattaccggtacaggctgttccggaagtccaatctgaagcccttcgagcgggacatctccaccgagatctatcaggccggcagcaagccttgcaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtacagaggtgccagtggccattcacgccgatcagctgacccctacttggcgggtgtactccacaggcagcaatgtgtttcagaccagagccggctgtctgatcggagccgagcacgtgaacaatagctacgagtgcgacatccccatcggcgctggcatctgtgccagctaccagacacagaccaacagcaggcggagagccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcagaacgtcgtgaaccagaacgcccaggcactgaacaccctggtcaagcagctgtcctccaacttcggcgccatctctagcgtgctgaacgatatcctgagcagactggaccctcctgaggccgaggtgcagatcgacagactgatcacaggcagactccagtctctgcagacctacgtgacccagcagctgatcagagccgccgagattagagccagcgctaatctggccgccaccaagatgtctgagtgtgtgctgggccagagcaagagagtggacttttgcggcaagggctaccacctgatgagcttccctcagtctgcaccacacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtggtcaacatccagaaagagatcgaccggctgaacgaggtggccaagaatctgaacgagagcctgatcgacctgcaagaactggggaagtacgagcagtacatcaagtggcctggcggatctggcagccctctggatatctctagcgagctgggcaaagtgaatcagagcctgtccaacgtggaagatctgattgaccagagcaaccagctgctgaacagagtgaaccccaacatcgtcaacaacaccgccatcatcgtgacaatcgtgctgctggtcctgctcgtgctgtggtgtctggccctgacaatcagcatcctgtatgtgtccaagcacgccgtgcggatgatcaagaccgtgcctaatccttacgtgatgcaggccaagtctcccggcagcgccacacagttttaa

SEQ ID NO: 22 - DNA, 3903 bp, unidentified
  source 1..3903: mol_type = other DNA
atgttcgtgtttctggtgctgctgcctctggtgtccagccagtgtgtgaacctgcggaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtgtactatcacaagaacaacaagagctggatggaaagcggcgtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattaccggtacaggctgttccggaagtccaatctgaagcccttcgagcgggacatctccaccgagatctatcaggccggcagcaagccttgcaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtacagaggtgccagtggccattcacgccgatcagctgacccctacttggcgggtgtactccacaggcagcaatgtgtttcagaccagagccggctgtctgatcggagccgagcacgtgaacaatagctacgagtgcgacatccccatcggcgctggcatctgtgccagctaccagacacagaccaacagcaggcggagagccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcagaacgtcgtgaaccagaacgcccaggcactgaacaccctggtcaagcagctgtcctccaacttcggcgccatctctagcgtgctgaacgatatcctgagcagactggaccctcctgaggccgaggtgcagatcgacagactgatcacaggcagactccagtctctgcagacctacgtgacccagcagctgatcagagccgccgagattagagccagcgctaatctggccgccaccaagatgtctgagtgtgtgctgggccagagcaagagagtggacttttgcggcaagggctaccacctgatgagcttccctcagtctgcaccacacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtggtcaacatccagaaagagatcgaccggctgaacgaggtggccaagaatctgaacgagagcctgatcgacctgcaagaactggggaagtacgagcagtacatcaagtggcctggcggatctggaagcgaactgggccagctgaacaacagcatcagctccgccaccgataacctgcagaaggcccaagagtccctgaacaagtctatcatccctgccgccacctccagctggctgatcatcctgctgtttgtgctcgtgtccatcagcctggtcatcggctgcatcagcatctacttcatctacaagcactccaccaccaacagaagccggaacctgtccagcgatatcatcagcaacccctatatccagaaggccaactga

SEQ ID NO: 23 - DNA, 3939 bp, unidentified
  source 1..3939: mol_type = other DNA
atgttcgtgtttctggtgctgctgcctctggtgtccagccagtgtgtgaacctgcggaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtgtactatcacaagaacaacaagagctggatggaaagcggcgtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattaccggtacaggctgttccggaagtccaatctgaagcccttcgagcgggacatctccaccgagatctatcaggccggcagcaagccttgcaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtacagaggtgccagtggccattcacgccgatcagctgacccctacttggcgggtgtactccacaggcagcaatgtgtttcagaccagagccggctgtctgatcggagccgagcacgtgaacaatagctacgagtgcgacatccccatcggcgctggcatctgtgccagctaccagacacagaccaacagcaggcggagagccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcagaacgtcgtgaaccagaacgcccaggcactgaacaccctggtcaagcagctgtcctccaacttcggcgccatctctagcgtgctgaacgatatcctgagcagactggaccctcctgaggccgaggtgcagatcgacagactgatcacaggcagactccagtctctgcagacctacgtgacccagcagctgatcagagccgccgagattagagccagcgctaatctggccgccaccaagatgtctgagtgtgtgctgggccagagcaagagagtggacttttgcggcaagggctaccacctgatgagcttccctcagtctgcaccacacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtggtcaacatccagaaagagatcgaccggctgaacgaggtggccaagaatctgaacgagagcctgatcgacctgcaagaactggggaagtacgagcagtacatcaagtggcctggcggaagcggcagcctggatatctctagcgagatcggcaacatcaacaacaccgtgaaccgcgtggaagatctgattcaccagagcgaggaatggctcgccaaagtgaaccctcacatcgtcaacaataccacactgatcgtgctgtgcgtgctgtctgccctggctgtgatttggctggctgtgctgaccgccatcatcatctacctgaggacaaagctgaaaacgatctcagccctggccgtgaccaataccatccagtctaacccctacgtcaaccagacaaagcgcgagagcaagttctga

SEQ ID NO: 24 - DNA, 10 bp, synthetic construct
  source 1..10: mol_type = other DNA
ttagaaaaaa

SEQ ID NO: 25 - DNA, 10 bp, synthetic construct
  source 1..10: mol_type = other DNA
acgggtagaa

SEQ ID NO: 26 - DNA, 21 bp, synthetic construct
  source 1..21: mol_type = other DNA
aaggtccaactctccaagcgg

SEQ ID NO: 27 - DNA, 23 bp, synthetic construct
  source 1..23: mol_type = other DNA
gtcctccttactatcagtccaca

SEQ ID NO: 28 - protein, 1327 aa, unidentified
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGSGYIPEAPRDGQAYVRKDGEWVFLSTFLGGSLGNVNNSISNALNKLEESNRKLDKVNVKLTSTSALITYIVLTIISLVFGILSLILACYLMYKQKAQQKTLLWLGNNTLDQMRATTKM

SEQ ID NO: 29 - DNA, 3984 bp, unidentified
  source 1..3984: mol_type = other DNA
atgtttgtttttcttgttttattgccactagtctctagtcagtgtgttaatcttagaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttggatgtttattaccacaaaaacaacaaaagttggatggaaagtggagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagattgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcgtggctccgcatccagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaaaatgtggtcaaccaaaatgcacaagctttaaacacgcttgttaaacaacttagctccaattttggtgcaatttcaagtgttttaaatgatatcctttcacgtcttgaccctcccgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagggatccggaagcggttatattcctgaagctccaagagatgggcaagcttacgttcgtaaagatggcgaatgggtattcctttctacctttttaggaggatcccttgggaatgtcaacaactcgatcagtaatgctttgaataagttagaggaaagcaacagaaaactagacaaagtcaatgtcaaactgactagcacatctgctctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtga